(19) United States
(12) Patent Application Publication
(10) Pub. No.: US 2023/0175940 A1
(43) Pub. Date: Jun. 8, 2023

(54) METHOD AND DEVICES FOR MEASURING VISCOSITY OF LIQUID FOODS

(71) Applicant: AKRIBIS SYSTEMS PTE LTD, Singapore (SG)

(72) Inventors: YONG PENG LEOW, Singapore (SG); GUOLIN XU, Singapore (SG); NIK BRIGHTWELL, Singapore (SG); HANCHEN WANG, Singapore (SG); YI MAO, Singapore (SG); KAI XIN NG, Singapore (SG)

(21) Appl. No.: 16/964,394

(22) PCT Filed: Jan. 3, 2020
(86) PCT No.: PCT/SG2020/050003

§ 371 Date: Jul. 23, 2020

(51) Int. Cl. G01N 11/02; G01N 33/02

(52) U.S. Cl. CPC G01N 11/02; G01N 33/02; A23L 19/09

(57) ABSTRACT

The device is a viscosity measurement indicator for measuring the viscosity of thickened liquids and liquid foods for Dysphagia patients using the IDDSI Framework wherein a sample of thickened liquids or liquid food is taken and introduced to the device to show the viscosity level based on the IDDSI Framework. The device may be a card or a cylinder.

Methods of measuring the viscosity of thickened liquids and liquid foods using the devices are also proposed.

A Kit for measuring the Viscosity of thickened liquids and liquid foods is also proposed. An accessory to help in the taking of viscosity measurement using the invention is also proposed.

### FIELD OF THE INVENTION

The present invention relates to a simple and cost-effective method and devices for measuring and indicating viscosity of thickened liquids and liquid foods to be consumed by persons having swallowing impairment (Dysphagia).

### BACKGROUND

Dysphagia is a symptom of impairment to the safety and/or efficiency of swallowing, frequently involves inappropriate timing, strength or coordination of muscle contraction leading to airway invasion or the accumulation of post-swallow residue. Swallowing impairment (or dysphagia) can develop with ageing or major illnesses, such as deconditioning after surgery, dementia, stroke, Parkinson's disease, head and neck cancer.

Dysphagia has negative effects on health, nutritional status, and quality of life. It also puts patients at risk of developing life-threatening aspiration pneumonia. To overcome the situations, thickened liquids are a widely-used intervention, based on the idea that slower flow of these thicker liquids provides the person with extra time in which to achieve airway closure as well as enabling better control of liquid flow by the tongue and other swallowing muscles.

Thickeners are used to thicken foods and fluids to various consistencies. Thickeners can help slow the transit of foods and fluids to allow more time to coordinate the swallowing process safely. This prevents the foods and fluids from entering the lungs accidentally with serious consequences resulting in health complications such as chest infections and even resulting in death due to choking or aspiration pneumonia.

In healthcare institutions and home care for Dysphagia patients, nurses or caregivers thicken drinks manually by mixing in powdered thickener every time drinks or medications are served. The Table below shows a standard for the preparation of thickened fluids involving 3 stages of viscosity and the corresponding consistency described in words:

| Description of | Description of Fluid to which a commercial |
|---|---|
| Thickened Fluid | thickener has been added to thicken consistency |
| Stage 1 | can be drunk through a straw |
| | can be drunk from a cup if advised or preferred |
| | leaves a thin coat on the back of a spoon |
| Stage 2 | cannot be drunk through a straw |
| | can be drunk from a cup |
| | leaves a thick coat on the back of a spoon |
| Stage 3 | cannot be drunk through a straw |
| | cannot be drunk from a cup |
| | needs to be taken with a spoon |

The amount of thickener required to thicken fluids to one of the desired 3 stages will depend on the type of thickener used. The companies manufacturing these thickeners provide specific information to state how much thickener is needed for particular fluids or will provide an overview guide of how many containers of thickener are required each 28 day prescription depending on the consistency needed. It is important not to confuse the types of thickeners as the starch based and gum based thickeners are two different products and are not interchangeable. Also the scoops provided by each manufacturer of these thickeners vary in size.

In 2002 the National Dysphagia Diet (NDD) was introduced. The NDD incorporated a scientific foundation for the dysphagia diet based on key rheological properties of foods and fluids. The NDD also included the consideration of physical forces of the chewing and swallowing process.

The varying standards in use of thickeners in preparing food and drinks for Dysphagia patients have made preparation of food and drinks very slow and subject to manual or visual checks for the correct viscosity for each Dysphagia patient. The problem is expected to multiply exponentially in many countries in view of increasing number of senior citizens who are living longer yet have declining health issues including Dysphagia. It is also foreseen that the problem of preparing thickeners is going to be exacerbated in nursing homes and hospitals in view of the increasing number of Dysphagia patients staying in each institution.

An initiative to introduce a common standard for Dysphagia Diet was made by the International Dysphagia Diet Standardisation Initiative Inc (IDDSI) which was founded in 2012. The work of the International Dysphagia Diet Standardisation Committee came to fruition in November 2015 with the final IDDSI framework. This framework selected an objective but practical measurement for liquids which could be used in kitchens and bedsides as well as laboratories.

The IDDSI flow test uses a standard 10 mL syringe as the “funnel”, Instead measuring the time required for a sample to flow through the syringe, the IDDSI flow test classifies consistency based on the volume of the residual liquid in the syringe after a period of 10 s flow.

The first type of thickeners being starch based and starch based thickeners were appropriate to use in catering/food production. In line with the development of standardisation for Dysphagia Diet, thickeners have also evolved over the years and factors such of appearance, ease of mixing, palatability and compliance has resulted in new ranges of thickeners that have been developed, which not only safe but delicious. These products include high protein snacks, hydration jellies, tasty puree dishes, and the smoothest thickener we've ever come across. As more people suffer from Dysphagia, more healthcare providers are offering more choices for patients with dysphagia, turning away from traditional thickeners, to include high protein snacks, puree and other more palatable foods. As such, there is a great need for a quick and low cost method to measure or indicate the viscosity of soft foods and drinks due to the diet requirements of dysphagia patients.

The measurement of viscosity for the Dysphagia Diet discussed herein includes liquid food and soft foods (“textured modified foods”) and Thickened fluids used in Dysphagia Management as defined by the IDDSI framework. Viscosity of the liquid foods is indicated by Levels, from Level 0 to Level 4.

### DISCUSSION OF PRIOR ART

The IDDSI has recommended an IDDSI Flow Test using a syringe which may contain up to 10 ml of liquid food. The liquid food which is to be checked for the right consistency has to be loaded with another 10 ml syringe. The standard 10 mL syringe acts as the “funnel”. Instead measuring the time required for a sample to flow through the syringe, the IDDSI flow test classifies consistency based on the volume of the residual liquid in the syringe after a period of 10 s flow.

The steps to be taken using the IDDSI Flow Test involved these steps:—

1. Remove plunger of the test syringe. Check that the nozzle of the test syringe is completely clear before use.
2. Cover nozzle of test syringe with finger and fill the test syringe with 10 ml of the fluid
3. Release the nozzle and start a timer.
4. Stop after 10 seconds. Use finger to stop flow of fluid from nozzle.
5. Remaining fluid is checked.

According to IDDSI Flow Test, the resulting levels are defined as:

Level 0: Thin (0-1 mL liquid remaining)

### Level 1: Slightly-Thick (1-4 mL)

### Level 2: Mildly Thick (4-8 mL)

### Level 3: Moderately Thick (8-10 mL)

Level 4: Extremely Thick (10 mL, no flow is released from the syringe)

Another quick method proposed by IDDSI is the “Fork Drip and Spoon Tilt Testing methods”. This method only uses fork or spoon to visually “test” the thickness of the drink. This quick method gives results which are very subjective.

Some benefits of the IDDSI Flow Test are as follows:
Repeatability
Contrast medium preparation
Easy to teach and learn
Cost-effective, inexpensive and accessible

The IDDSI Flow Test still suffers from some disadvantages:—
Not easy to operate as some training is required
Large amount of sample (10 mL) needed
High costs of syringe
Time consuming in preparing and time consuming in cleaning the syringes (if re-used)

Another Test for determining the Drink Thickness Level based on the IDDSI Framework had been proposed. A Quick Easy Accurate Drink Thickness Test Kit has been proposed by Viscgo Limited (website: https://viscgo.com/viscgo). The kit consists of 1 or 2 sticks (for use to indicate Level 1, Level 2, and Level 3) that have been calibrated by Associate Professor Ben Hanson of University College London, Board Member of IDDSI, designer of the IDDSI Flow Test, to ensure they accurately adhere to the IDDSI Framework.

The Viscgo Test Kits come in various types such as Level 1 Test Kit, Level 2 Test Kit and Level 3 Test Kit. The Dysphagia patient or the care giver would use the appropriate Viscgo sticks to check every drink is at the prescribed consistency and level. The Viscgo drink thickness test is simple as the Viscgo stick is placed upright in the liquid food. Due to the viscosity of the liquid foods, the stick will either stand (for a while) or fall depending on the thickness of the liquid and the dimensions and mass of the stick.

Each Viscgo drink thickness test stick is designed to fall within 10 seconds if a drink is below a certain level of thickness and stand for at least 10 seconds if a drink is above a certain level of thickness. Although this the kit is simple in structure, it suffers the following drawbacks:
1) Depth of the tested drink must be between 6.5 cm and 7.5 cm deep.
2) Both two measuring sticks (for Level 1 and Level 2) or one measuring stick (for Level 3) must stand upright in the drink before timing, which is not easy to achieve for aged people whose hands are shaking.
3) Time sensitive, must check the whether the stick or sticks fall down or not in 10 seconds.
4) Unable to measure thickness of Level 0 and Level 4.

Furthermore, the Viscgo drink thickness test also require some skill in judging the viscosity and corresponding Level under the IDDSI Framework. Based on the inventors' understanding of how the Viscgo Test works, it would seem a substantial volume of liquid food has to be used to carry out the test each time.

The Inventors applaud the common standard for indicating viscosity of thickeners and Liquid Foods for Dysphagia Diet as proposed under IDDSI Framework. They have noted that there are drawbacks to the current tests for viscosity in management of the Dysphagia Diet. Other types of viscosity measurement devices are either too costly for the simple objective to be achieved or too difficult to carry out requiring some skill and training in use of the tests.

The IDDSI Flow Test and Viscgo Sticks Method have their advantages. However any test to check the viscosity of liquid foods and thickeners are at the appropriate level (under the IDDSI System) has to be carried out a number of times daily for each Dysphagia patient in order to meet daily requirements for sufficient fluids and sufficient calories intake, which is definitely more than the usual three times for meals for a normal healthy adult.

The inventors have examined other types of viscosity testing devices which have been proposed but each viscosity testing device or method has been found to be inappropriate and unsuitable for management of Dysphagia patients using the Dysphagia Diet.

U.S. Pat. No. 4,741,200 proposes a piezoelectric element energized to exhibit thickness-shear mode vibrations to measure the viscosity of liquids, Although this type of sensor is simple, but it requires complicated circuit for actuating and sensing. In the meantime, it is not easy to clean after using due to brittle characteristic of piezoelectric element. On the other hand, piezoelectric element sensor is expansive and cannot be used for disposable application.

CN208150962 is an utility model which proposes use of a MEMS chip for measuring liquid viscosity. The MEMS chip includes the substrate, be equipped with the liquid storage tank on this substrate, lie in this liquid storage tanktop the cantilever beam, set up at liquid runner of this liquid storage tank one side and the drops of liquid outlet who is linked together with the liquid runner, wherein, be equipped with on this cantilever beam and be used for a static driven driving electrode, still be equipped with on the substrate with the 2nd driving electrode that a driving electrode corresponds, the liquid storage tank is used for accepting the volume of awaiting measuring liquid, the viscosity that the cantilever beam is used for measuring this liquid changes. The utility model discloses a MEMS chip structure is simpler, and manufacturing cost is lower, and production efficiency is higher to but wide application in technical field such as medical care and public health, oil, chemical industry, metallurgy, national defense. In addition, adopt the utility model discloses a MEMS chip can detect the precision, and is higher with the IC compatibility, more is fit for use individualized and domestic. Although using MEMS fabrication technology can reduce the cost of the chip device significantly, however, this MEMS chip is still too expansive for disposable application. The complicated actuating and sensing electrical circuit may the whole measurement device expensive too.

U.S. Pat. No. 6,412,338 proposes a viscometer for sensing or characterizing the stress required to shear a liquid at a given rate including a pair of members coaxially mounted to relative rotation. Between the members is an annular gap defining a flow path for the fluid. The flow path is configured such that during differential rotation of the members, the fluid is caused to flow through the annular gap that is a function of the differential rotation and the viscosity of the fluid, A sensor measures the torque or torque equivalent required to achieve such differential rotation between the members.

Similar to U.S. Pat. No. 6,412,338, CN206740582 is an utility model which discloses a rotational viscometer, which comprises a housing, the inside of casing is provided with the division board, the top of division board is provided with the measuring chamber, the bottom of division board is provided with driving chamber, the inside of measuring chamber is provided with the viscosity detection piece, and provided with passive magnet on the viscosity detection piece, one side of measuring chamber is provided with the fluid entry, the opposite side of measuring chamber is provided with the fluid issuing, driving chambers inside is provided with magnetic force conduction piece, provided with the initiative magnet on the magnetic force conduction piece, the bottom of magnetic force conduction piece is provided with rotary main shaft, the passive magnet that sets up, the initiative magnet, first magnet and second magnet not only can divide actuating mechanism to be opened with measurement medium, and measure the fluid viscosity changes as current signals, can result in more accurate fluidic viscosity.

Both technologies from U.S. Pat. No. 6,412,338 and CN206740582 using rotational technique for viscosity measurement. Their measuring accuracy is high. However, both technologies are very complicated and require very large amount of sample during measuring process. They are not suitable for drink thickness measurement.

U.S. Pat. No. 4,517,830 describes an apparatus for detecting the viscosity of blood comprises a hypodermic syringe including a transparent hollow tube, a needle secured to one end of the tube and a plunger in the tube for creating a vacuum for extracting a predetermined volume of blood and depositing the blood in the transparent hollow tube for immediately performing internal viscosity measurement without substantial change in temperature and without any chemical change (e.g. oxidation) in the blood sample. An apertured weight member within the transparent hollow tube is movable by gravity at a rate which is a function of the viscosity of the blood and indicia means, visible on the side of the transparent tube indicates the rate of movement of the apertured weight member through the predetermined volume of blood as a measure of the viscosity of the blood in the tube. This technology has advantage such as without complicated electrical circuit and easy to use. However, this technology requires a very complicated plunger structure. This will make the whole device very expansive in fabrication cost and cannot be disposable. On the other hand, the whole device is difficult to clean for reuse. Hence it is difficult for apply this technology for drink thickness measurement.

The prior art documents all suffer from either unsuitability for a quick check on viscosity or relatively high costs.

In view of the increasing number of Dysphagia patients in many countries, the inventors foresee that caregivers in hospitals and nursing homes would not be able to cope with the volume and number of daily feedings of Dysphagia patients.

The inventors are of the view that what most health care institutions and hospitals and even for home-based dysphagia patients desire is a quick, simple and efficient yet low cost method of viscosity measurement for thickeners and liquid foods as well as a simple and reliable viscosity measurement device for carrying out the viscosity measurement method quickly. The viscosity measurement method and device should not be too demanding in terms of dexterity and skill for such a routine and frequent act in preparing liquid foods with the right level of viscosity for a Dysphagia patient. It must be borne in mind that the preparatory work before measurement and cleaning work after measurement are important factors in considering suitability of any viscosity measurement device. Finally, it must be borne in mind that any viscosity measurement method proposed has to carried out daily many times for Dysphagia patients as any food or drink to be swallowed must meet the prescribed viscosity level.

In support of this Framework, the inventors have proposed a simpler and quicker yet effective approach to checking the viscosity of Thickeners and Liquid Foods according to the IDDSI Framework and which would benefit both Dysphagia patients and Healthcare providers such as Hospitals, Nursing Homes and individual car givers which would be described hereinafter.

### SUMMARY OF THE INVENTION

This summary introduces, in a simplified form, concepts that are further described below in the detailed description. This summary is not intended to identify key or essential features of the claimed subject matter, and is not intended to be used as an aid in determining the scope of the claims.

A first objective is a viscosity measurement indicator device for measuring the viscosity of thickened liquids and liquid foods for Dysphagia patients using the International Dysphagia Diet Standardisation Initiative (IDDSI) Framework comprising three layers, namely a first layer, a measuring membrane layer and a 3rd layer, the 1st layer, the measuring membrane layer and the 3rd layer arranged or combined together to form the device, wherein a sample of thickened liquids or liquid food is taken and introduced to the viscosity measurement indicator device to show the viscosity level based on the IDDSI Framework.

Preferably the viscosity measurement indicator device is a card.

Alternatively the viscosity measurement indicator device is a cylinder.

Preferably the layer on top of the measuring membrane has an opening to allow introduction of the sample of thickened liquids or liquid food for measurement of viscosity thereof.

Preferably the measuring membrane of the viscosity measurement indicator device contains a hydrophilic substance which changes colour upon detection of water in the sample of thickened liquids or liquid food to be measured.

Preferably the viscosity measurement indicator device has a 4th layer being a hydrophilic substance which changes colour upon detection of water in the sample of thickened liquids or liquid food to be measured.

Preferably the length of the measuring membrane of the viscosity measurement indicator device has Viscosity Indicators based on the IDDSI Framework calibrated to indicate a viscosity level from Level 0 to Level 4.

Preferably the measuring membrane of the viscosity measurement indicator device is made from paper.

Alternatively the measuring membrane of the viscosity measurement indicator device is made from cloth.

Alternatively the measuring membrane of the viscosity measurement indicator device is made from pure borosilicate glass.

Alternatively the measuring membrane of the viscosity measurement indicator device is made from nitrocellulose membrane.

Alternatively the cloth used in the measuring membrane of the viscosity measurement indicator is cellulose fiber.

Alternatively the cloth used in the measuring membrane of the viscosity measurement indicator device is alpha cotton cellulose.

Alternatively the cloth used in the measuring membrane of the viscosity measurement indicator device is linter fibers or cotton linter.

Preferably the cylinder of the viscosity measurement indicator device has a 1st layer being an outer surface and 3rd layer being an inner surface, and the inner surface of the cylinder is treated with a layer of hydrophilic substance and the length of the cylinder has Viscosity Indicators based on the IDDSI Framework calibrated to indicate a viscosity level from Level 0 to Level 4.

Alternatively the inner surface of the cylinder of the viscosity measurement indicator device is lined with a viscosity measurement layer treated with a hydrophilic substance and the length of the cylinder has Viscosity Indicators based on the IDDSI Framework calibrated to indicate a viscosity level from Level 0 to Level 4.

Preferably the viscosity measurement layer of the viscosity measurement indicator cylinder contains a hydrophilic substance which changes colour upon detection of water in the sample of thickened liquids or liquid food to be measured.

Preferably the viscosity measurement layer of the viscosity measurement indicator cylinder is made from paper.

Alternatively the viscosity measurement layer of the viscosity measurement indicator cylinder is made from cloth.

Alternatively the viscosity measurement layer of the viscosity measurement indicator cylinder is made from pure borosilicate glass.

Alternatively the viscosity measurement layer of the viscosity measurement indicator cylinder is made from nitrocellulose membrane.

Alternatively the cloth used in the viscosity measurement layer of the viscosity measurement indicator cylinder is cellulose fiber.

Alternatively the cloth used in the viscosity measurement layer of the viscosity measurement indicator cylinder is alpha cotton cellulose.

Alternatively the cloth used in the viscosity measurement layer of the viscosity measurement indicator cylinder is linter fibers or cotton linter.

A second objective of the invention is a method to measure the viscosity of thickened liquids and liquid foods for Dysphagia patients using the International Dysphagia Diet Standardisation (IDDSI) Framework, comprising the steps of (i) introducing a sample of thickened liquids or liquid food to the viscosity measurement indicator device and (ii) observing dispersion action of the sample in the measurement membrane to indicate the viscosity level based on the IDDSI Framework.

Preferably the step includes having the sample of thickened liquids or liquid food in the viscosity measurement cylinder with marker line thereon is dipped into a sample of thickener liquid or liquid foods and said marker line is aligned with the liquid level, and after several seconds, observing dispersion action of the thickened liquid upwards the cylinder to indicate the viscosity level based on the IDDSI Framework.

A third objective of the invention is a kit to measure and adjust the viscosity of thickened liquids and liquid foods for Dysphagia patients using the International Dysphagia Diet Standardization (IDDSI) Framework using the viscosity measurement indicator device of the invention, a pipette to introduce a sample of said thickened liquids and liquid foods into the viscosity measurement indicator card and a spoon for measuring water to be added to the thickened liquids and liquid foods for adjustment of the viscosity thereof.

Alternatively the kit to measure and adjust the viscosity of thickened liquids and liquid foods for Dysphagia patients using the International Dysphagia Diet Standardization (IDDSI) Framework comprises the viscosity measurement indicator device of the invention, a liquid dropper to introduce a sample of said thickened liquids and liquid foods into the viscosity measurement indicator card and a spoon for measuring water to be added to the thickened liquids and liquid foods for adjustment of the viscosity thereof.

A fourth objective of the viscosity measurement indicator device for measuring the viscosity of thickened liquids and liquid foods for Dysphagia patients using International Dysphagia Diet Standardisation Initiative (IDDSI) Framework is a device holder with an alignment hole, said device holder adapted to fit and hold onto the sides of the viscosity measurement card so that the alignment hole is directly over the opening of the measurement membrane for easy introduction of the thickened liquids and liquid foods onto the measurement membrane.

### BRIEF DESCRIPTION OF THE DRAWINGS

For a better understanding of the invention, its advantages, and the objects attained by its use, reference should now be made to the accompanying drawings. The accompanying drawings illustrate one or more embodiments of the invention and together with the description herein, serve to explain the workings and principles of the invention. The diagrams by no means restrict the invention to only what is shown.

The invention is a quick, simple and efficient yet low cost method of measuring or indicating the thickness of thickened liquid foods for the Dysphagia Diet for Dysphagia patients based on Level 0 to Level 4 under the IDDSI Framework. The inventive method also proposed a simple, low cost and reliable viscosity measurement device or viscosity level indicator (based on the IDDSI framework) for carrying out the viscosity measurement method quickly. The viscosity measurement indicator device may have the ability to show presence of water with or without color change when thickened liquid foods are introduced into the viscosity measurement indicator device. Furthermore, the invention provides a systematic tool for adjusting the thickness of the drink after thickness measurement according the viscosity measurement using the device.

The viscosity measurement indicator may be in the form of an elongated strip of card or rectangular piece of treated paper calibrated to correspond to the measurement of viscosity of liquid foods and thickeners based on the scale of Level 0 to Level 4 proposed by the IDDSI. Alternatively the viscosity measurement gauge may be in the form of a cylinder with markings calibrated to correspond to the measurement of viscosity of liquid foods and thickeners based on the scale of Level 0 to Level 4 proposed by the IDDSI.

Since the determination of viscosity of the Liquid foods and thickeners are based on the IDDSI Framework, the method and device would be easily adopted by hospitals and care givers due to familiarity or training with the IDDSI system.

The use of this inventive method and device may involve a card or other type of indicator or gauge (which may be in the form of a cylinder). Whichever the embodiment used, the steps in using this inventive method and device to indicate or measure viscosity levels based on the IDDSI Framework would not be too demanding on users in terms of dexterity and skill required for such a routine and frequent exercise used daily to determine the suitability or swallowability of liquid food for a Dysphagia patient.

FIG. 1 is a perspective view of a 1st embodiment of the viscosity measurement indicator device in the form of a card with indicator levels for measuring the level of viscosity using a specific amount of drink volume drawn by a pipette based on the IDDSI standard.

FIG. 2 is a cross section of the components of viscosity measurement indicator device in the form of the card of FIG. 1. There are two configurations. The first configuration is shown in FIG. 2 (a), in which the measurement member consists of only one layer. This measurement member can be paper of different types, clothing of different type (linter fibers and cotton linter), non-woven cloth, pure borosilicate glass, nitrocellulose membrane, cellulose fiber and alpha cotton cellulose. The measurement member may contain a color indicator for indicating existence of water when the measuring member absorbs water. The color indicator can be a chemical compound such as phenolphthalein, cupric sulfate and etc. which is known in the art.

The second configuration of the viscosity measurement indicator card of FIG. 1, is shown in FIG. 2 (b). The measurement member has two or more layers. For example, one layer contains alkaline, such as dried sodium hydroxide, the other layer contains a chemical compound which changes colour in the presence of water such as phenolphthalein and other chemical compounds known for such property. When the sample contains water, the measurement member absorbs water, the chemical compounds from the two layers reacts and the colourless water changes to colour, for easy viewing.

FIG. 3 shows first embodiment of the viscosity measurement indicator device in the form of card with indicator levels to test the viscosity levels of liquid food and the spread of liquid food. The indicator markings indicate a range of viscosity from Level 0 to Level 4, based on the IDDSI Standard.

FIG. 4 shows a system for drink thickness measurement and adjustment of viscosity of thickened liquids and liquid foods. It consists of a viscosity measurement indicator device in form of a card, a disposable or reusable drink collecting pipette, a liquid measuring cup and a spoon with 2 or more measuring volumes.

FIG. 5 is a perspective view of a 2nd embodiment of the viscosity measurement indicator device in the form of a card with indicator levels for measuring the level of viscosity of drink. It uses a narrow indication window for easy reading of the results.

FIG. 6 is a cross section of the components of viscosity measurement indicator device in the form of the card of FIG. 5. Its construction is similar to first embodiment (as shown in FIG. 2) Its measurement member maybe one layer, or two or more layers. Each layer may or may not contain a chemical compound to indicate presence of water through colour change indication.

FIG. 7 is a perspective view of a 3rd embodiment of the viscosity measurement indicator in the form of another card with indicator levels for measuring the level of viscosity based on the IDDSI standard. In this embodiment, to increase the length of drink spread for higher measurement sensitivity, the measurement member may be treated with a chemical compound which is hydrophilic or hydrophobic.

FIG. 8 is a cross section of the components of viscosity measurement indicator similar in structure and form to the card of FIG. 6. Its construction is similar to the first embodiment (as shown in FIG. 2). The viscosity measurement member may be one layer or may consist of two or more layers.

Each layer may or may not contain a chemical compound to indicate presence of water through colour change indication.

FIG. 9 shows a device holder with an alignment holder for loading a drink sample onto the viscosity measurement device.

FIG. 10 shows the procedure in measuring the viscosity of liquid foods (based on the IDDSI System) using the measurement indicator in the form of another viscosity measurement indicator which is in the form of a cylinder.

FIG. 11 shows how e viscosity measurement indicator device in the form of a cylinder is used,

FIG. 12 shows the measurement results for drink of thickness 0 to thickness 4, according to IDDSI standard.

FIG. 13 shows the steps in manufacturing a roll of viscosity measurement cards.

### DETAILED DESCRIPTION OF EMBODIMENTS OF THE INVENTION

The present invention relates to a viscosity measurement device to measure the viscosity of liquid food and thickeners for diet management of Dysphagia Patients using the IDDSI system of Level 0 to Level 4. The device may come into various forms or embodiments such as elongate card or rectangular card or gauge or cylinder.

A pipette is proposed to be used to take up a small amount of liquid food sample for the viscosity measurement. Alternatively a liquid dropper could be used. The pipette could be re-used if cleaned properly after use and the usual hygienic steps taken to ensure the pipette is sterile.

Alternatively a liquid dropper may be used. The liquid dropper may also be re-used if cleaned properly after use and the usual precautionary steps on hygiene and cleanliness taken. A reusable pipette is recommended to lower costs especially when there is a high volume of liquid food and thickeners to be prepared e.g. in a hospital or nursing home.

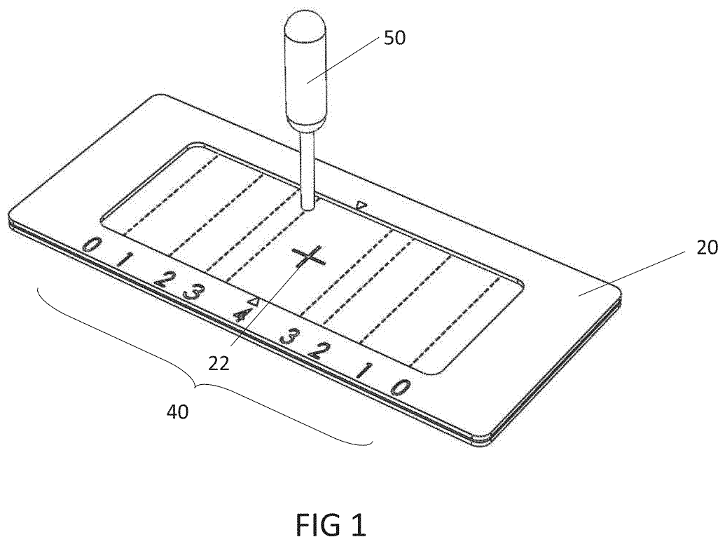

FIG. 1 is a perspective view of a 1st embodiment of the viscosity measurement indicator card (20) for measuring the level of viscosity based on the IDDSI standard. It includes an open window (22) for loading a sample of thickener or liquid foods using a pipette (51) for measuring viscosity, and a indicator with a series of lines with numbers (40) calibrated on IDDSI Standard for indicating viscosity levels of the sample.

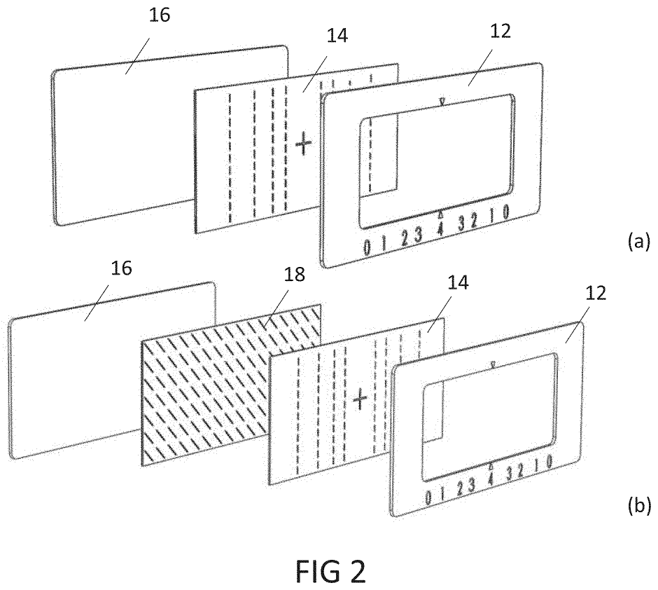

FIG. 2 is a cross section of the components of viscosity measurement indicator in the form of the card of FIG. 1.

With reference to FIG. 2, the viscosity measurement card consists of at least three layers. A measuring member layer (44) is contained within a measuring member holder layer (12). Two possible configurations are discussed herein. The first configuration is shown in FIG. 2 (a). It consists of three layers, a 3rd layer being a base layer (16), a 2nd layer being a measuring membrane (14) and a 1st layer being a measuring membrane holder (12). The measuring membrane (14) is contained within a measuring membrane holder (12). The 3rd layer is a base layer (16) on which the second layer (14) being the measuring membrane is affixed onto. The measuring membrane (14) is hydrophilic in nature while the measuring membrane holder (12) is hydrophobic in nature. The centre of the measuring membrane (14) has an inlet or opening (22) for a liquid food sample to be introduced using a pipette or a dropper. The length of the measuring membrane holder is marked with several Viscosity indication marks (40) using the IDDSI system which is from Level 0 to Level 4. These Viscosity indication marks (40) have been calibrated to correspond to the viscosity of liquid food based on the IDDSI system. This measurement member can be paper of different types, clothing of different type (linter fibers and cotton linter), non-woven cloth, pure borosilicate glass, nitrocellulose membrane, cellulose fiber and alpha cotton cellulose. The measurement member (14) may contain color indicator for indicating existing of water when the measuring member absorbs water. The color indicator can be chemical compound such as phenolphthalein, cupric sulfate and others which is known in the art. The second configuration, is shown in FIG. 2 (b). The measurement member (14) has two or more layers. For example, one layer contains alkaline, such as dried sodium hydroxide, the other layer contains phenolphthalein. When the measurement member comes into contact with water, non-color water becomes color for easy viewing.

The measuring membrane (14) being hydrophilic in nature may be paper of different types, clothing of different type (linter fibers and cotton linter), non-woven cloth, pure borosilicate glass, nitrocellulose membrane, cellulose fiber and alpha cotton cellulose. It can be a flat/non-flat surface with hydrophilic property

The thickness of the measuring membrane with thickness ranges from 0.05 mm to 5 mm, preferably range from 0.1 mm to 0.5 mm.

The measuring membrane holder being hydrophobic in nature may be thin film plastic material or thin paper which is able to hold the measuring membrane.

The base layer has some adhesive for affixing the measuring membrane and measuring membrane holder may use pressure sensitive glue, UV glue, thermal bond glue or using thermal diffusion bonding for combining the three layers together.

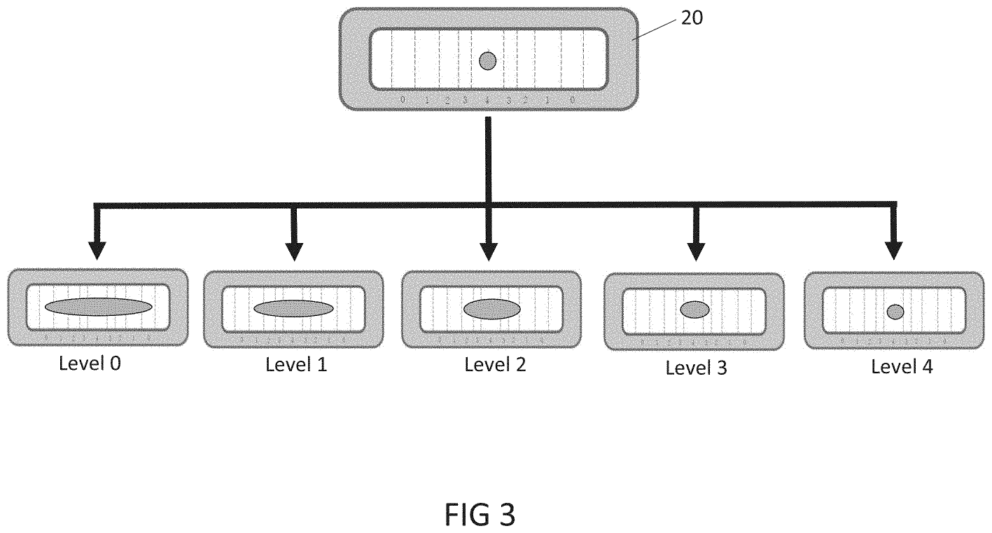

The measurement of viscosity of liquid food using the IDDSI system is described by reference to FIG. 3.

The viscosity measurement indicator card may come in rolls or blister packs. A card is detached from the roll or taken out from the blister pack. Using a pipette (50), a drop of liquid food which has been prepared and to be tested for viscosity under the IDDSI system is then withdrawn from the liquid food. The volume of the liquid food can range from 50 μL to 500 μL. The drop of liquid food is then dropped onto an opening at the centre of the viscosity measurement card. Alternatively a liquid dropper may be used to take up a sufficient quantity of the liquid food and the liquid food in the liquid dropper then ejected into an opening in the centre of the viscosity measurement card.

The food preparer then waits for 10 seconds and observe the dispersion action of the sample as the drop of liquid food spreads on the center of the viscosity measurement card. Since the viscosity measurement indicator card is calibrated with indicator markings to show Level 0 to Level 4, where the drop had spread to, would be the indicator of the Level of viscosity of the liquid food. The food preparer is thus able to see visually whether the liquid food is at the desired Viscosity Level for the Dysphagia patient.

FIG. 3 shows the results of viscosity measurement using the first embodiment of the viscosity measurement device in the form of card (20) with indicator levels to test the viscosity levels of liquid food and the spread of liquid food along the measuring membrane through dispersion action of the sample. The indicator markings (40) indicate a range of viscosity from Level 0 to Level 4, based on the IDDSI Standard. The procedure for measurement of viscosity of the liquid food based on the IDDSI system is to take a drop of liquid food which has been prepared and test it to determine the viscosity level of the liquid food based on the IDDSI System. A sample of the liquid food is then dropped from the pipette (50) onto the centre of the viscosity measurement card (20). The food preparer then waits ten seconds to see the dispersion action of the sample by observing how far the liquid food has spread along the measurement membrane (14). By visually observing how far the liquid food has spread along the measurement membrane as checked against the Level indicators, the food preparer would be able to confirm whether the liquid food meets the desired level under the IDDSI system for the Dysphagia patient.

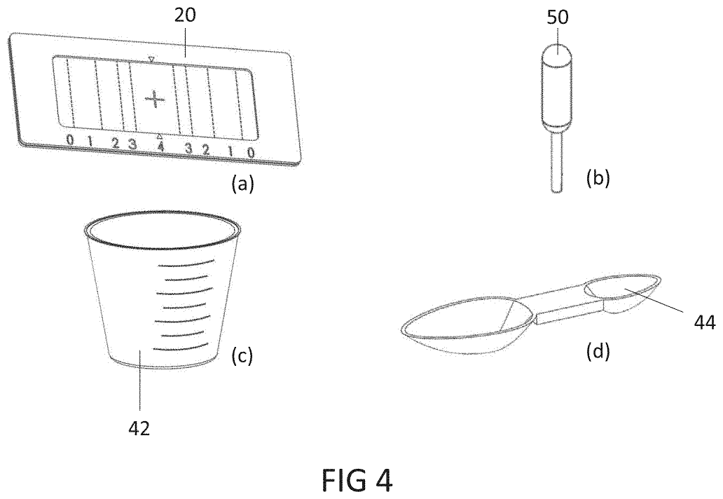

FIG. 4 shows the system for drink thickness measurement and adjustment of viscosity of thickened liquids and liquid foods. It consists of a viscosity measuring indicator card (20); a pipette (50), a liquid measuring cup (42) with volume indicator and a spoon (44) with capacity of two different volumes. After an user had measured the thickness of thickener drink, he/she can adjust the thickness of the drink according to the viscosity level preferred/required by doctor and or nurse. For example, if the measured thickness of the drink is too thick, according to the sample results, certain amount of water or original drink can be measured by the liquid measuring cup and added into the drink mixture to reduce the thickness. On the contrary, if the measured drink is too thin, certain amount of thickening powder can be measured by the spoon and added into the drink mixture.

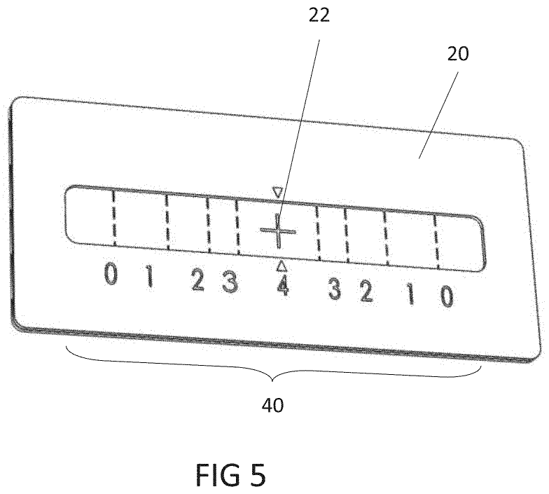

FIG. 5 is a perspective view of a 2nd embodiment of the viscosity measurement indicator device in the form of a card (20) with indicator levels (40) for measuring the level of viscosity of drink or liquid food. It uses a narrow indication window (22) to allow for increase in the spread length of the thickened drink and the indication marks are closer for easy reading of the results.

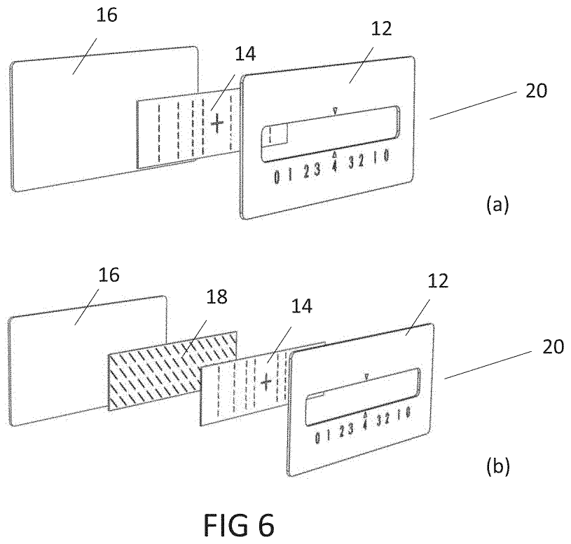

FIG. 6 is a cross section of the components of viscosity measurement indicator device in the form of the card of FIG. 5. Similar to the 1st embodiment, its measurement member may consist of one layer (FIG. 6(a)), two or more layers (FIG. 6(b)). Each layer may or may not contain a chemical compound to indicate existence of water in the sample.

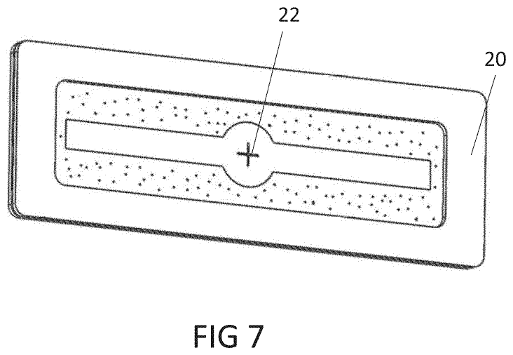

FIG. 7 is a cross section of the components of viscosity measurement indicator device of the 3rd embodiment as shown. In this embodiment, for increasing the length of drink spread for higher measurement sensitivity, the measurement member may be treated with a chemical compound which is hydrophilic and/or hydrophobic.

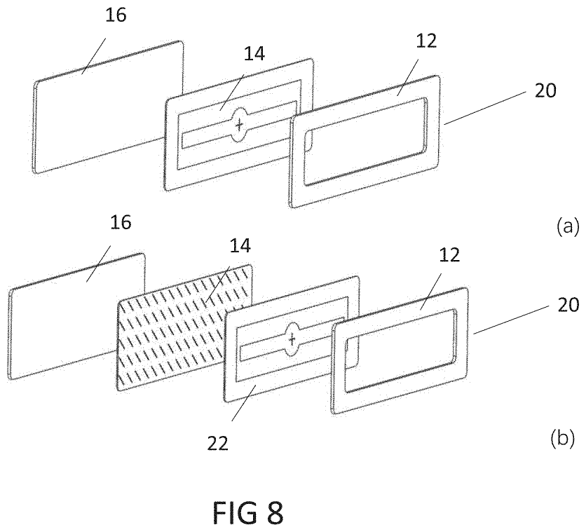

Referring to FIGS. 7 and 8, the 3rd embodiment is similar in construction to the 1st embodiment. The 3rd embodiment consists of three or more layers as shown in FIG. 8 (a) and (b). A measuring membrane (14) is contained within a measuring membrane holder (12). The 3rd layer is a base layer (16) on which the second layer (14) being the measuring membrane is affixed onto. The measuring membrane (14) is hydrophilic in nature while the measuring membrane holder (12) is hydrophobic in nature. The centre of the measurement member has an inlet or opening (22) for a liquid food sample to be introduced. Its construction is similar to that of the first embodiment. The measurement member may be one layer, two or more layers. Each layer may or may not contain a chemical compound to indicate presence or absence of water. The length of the measuring membrane holder is marked with several Viscosity indication marks (40) using the IDDSI system which is from Level 0 to Level 4.

The workings of the 3rd embodiment are similar to the workings of the 1st embodiment and would not be described again, for the sake of brevity.

The workings of the 3rd embodiment are also similar to the workings of the 1st embodiment and would not be described again, for the sake of brevity.

In all embodiments, a pipette or liquid dropper is used to introduce liquid food into an opening in the hydrophilic membrane. The pipette or liquid dropper would form part of the kit for measuring viscosity of liquid foods and thickeners for Dysphagia patients. Use of the pipette or dropper would ensure hygienic standards are kept when preparing liquid foods and thickeners for Dysphagia patients. The pipette and liquid dropper may be re-used if washed and sterilised according to the requirements of hospital and nursing homes.

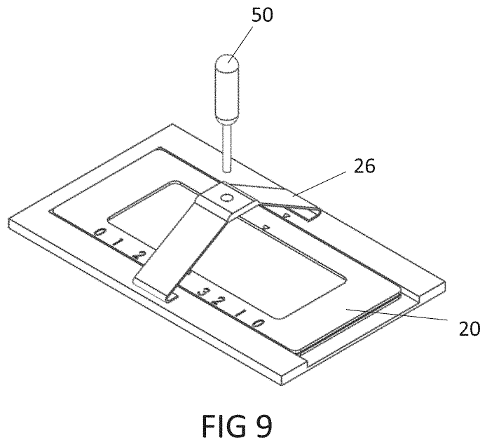

FIG. 9 shows a device holder (26) with alignment hold for loading the drink on to the viscosity measurement card (20). The device holder (26) will have guides to allow an user to set the holder directly over the opening (22) of the measuring membrane. This device holder will make it easy to people such as caregivers who may not be trained to load a sample of the thickened drink into the opening of the viscosity measurement indicator card using a pipette or a dropper (50) to measure the viscosity of thickeners and liquid foods.

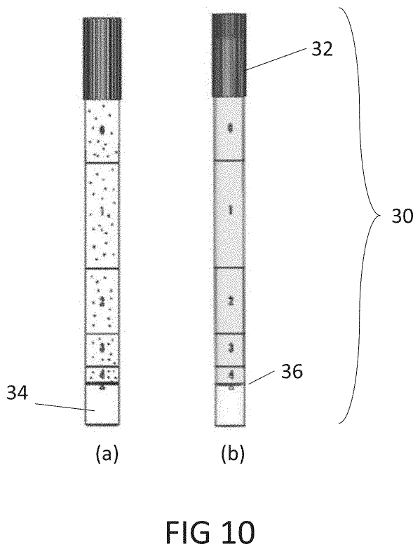

FIG. 10 shows another embodiment of the viscosity measurement indicator in the form of a cylinder. Two variants of this cylindrical viscosity measurement indicator are shown. The cylinder (30) operates in the same manner as in the other card indicators. The cylinder (30) is dipped into the liquid food for Dysphagia patients to be tested for viscosity before being served. The top of the cylinder has a holder (32) for the user to hold. The other end of the cylinder (34) is then dipped into the liquid food. A marker (36) is shown at this dipping end to show how far the tip should be dipped into the liquid food. After the recommended time (about 10 seconds) observing the dispersion action of the liquid food up the cylinder, the cylinder is withdrawn. The user then note at which viscosity indication mark the liquid food has gone up the cylinder (through this dispersive action), to tell the viscosity level of the liquid food (based on the IDDSI System).

The variants of this cylinder viscosity measurement indicator are as follows:—
1) the inner surface of the cylinder is treated with a layer of hydrophilic substance as shown in FIG. 10 (a)
2) similar to the configuration of card in FIG. 2. There is a layer of viscosity measurement indicator paper is on the inside surface of the cylinder FIG. 10 (b)

In the case of viscosity measurement cylinder with the viscosity measurement indicator paper, the treated paper is lined on the inside surface of the cylinder. This treated paper has been calibrated to correspond to the Level 0 to Level 4. These Viscosity indication marks have been calibrated to correspond to the viscosity of liquid food based on the IDDSI system:—
Level 1=below 50 mPa·s @50/s
Level 2=51-350 mPa·s @50/s
Level 3=351-1750 mPa·s @50/s
Level 4=1750-350 mPa·s @50/s

The viscosity measurement cylinder may be made of plastic, coated paper, glass and other material with certain strength.

The inner surface of the viscosity measurement cylinder may be treated with a layer of hydrophilic substance and outer surface calibrated to the viscosity of liquid foods based on the IDDSI System.

For example, the hydrophilic substance may change colourless water to pink colour by one layer containing an alkali and the other layer containing phenolphthalein.

The second variant of the viscosity measurement cylinder may have the inner surface lined with an additional layer of a paper treated with a hydrophilic substance and calibrated to the viscosity of liquid foods based on the IDDSI system. Again, the hydrophilic substance may change colourless water to pink colour by one layer containing an alkali and the other layer containing phenolphthalein.

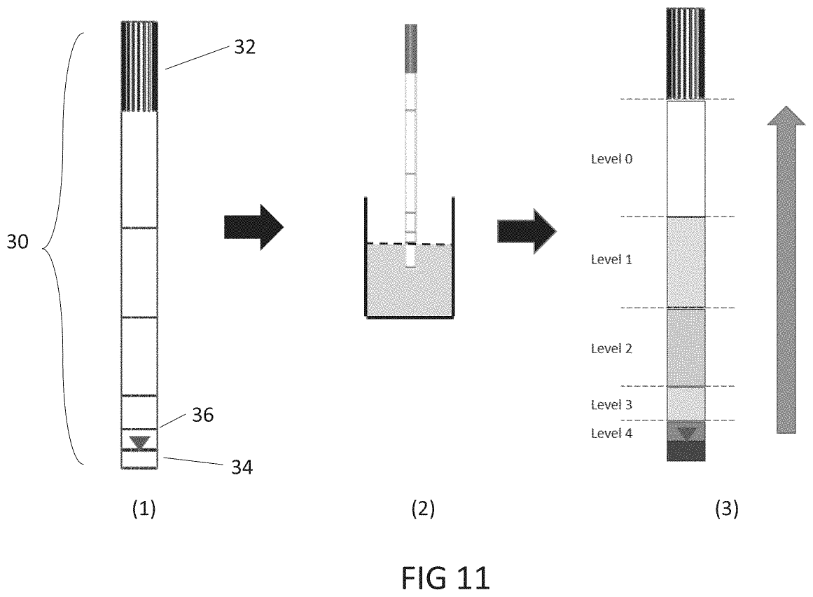

FIG. 11 (a), (b) and (c) shows the procedure in measuring the viscosity of liquid foods (based on the IDDSI System) using the viscosity measurement indicator in the form of the cylinder of FIG. 10.

In the description of the viscosity measurement indicator device using a card or strip, a sample of the liquid food has to be introduced into an opening in the card or strip. The obtaining of the sample would be carried out with a pipette (50) or a liquid dropper. In the case of the cylinder embodiment, the cylinder (30) has to be aligned upright and then dipped into the liquid food as shown in FIG. 11(b). A marker (36) near the dipping end of the cylinder (which is to be dipped into the thickened liquid or liquid food) would guide the food preparer. The surface of the liquid food has to be aligned to the marker (36) near the dipping end of the cylinder. The liquid food would travel up the end of the cylinder through dispersion action. After some time (10 seconds), the user would be able to determine how far the liquid food has crept up the viscosity level indicators on the cylinder. Since the viscosity level indicators in the cylinder has been calibrated using the IDDSI system, the viscosity level to which the liquid food had travelled up the cylinder may be read to indicate the viscosity level of the thickener or liquid food.

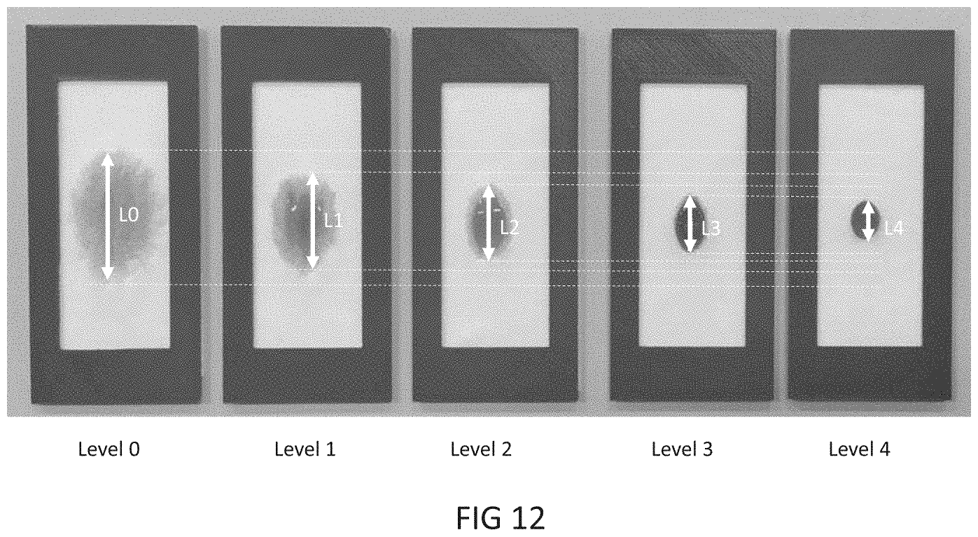

FIG. 12 shows the spread of liquid food across the viscosity measurement indicator card which would be calibrated to corresponds to viscosity levels of liquid food (from Level 0 to Level 4).

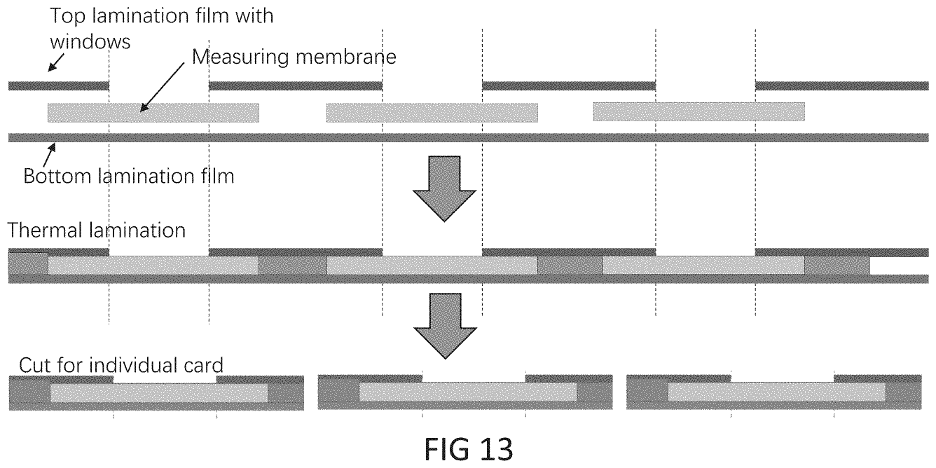

FIG. 13 shows the steps in manufacturing a roll of viscosity measurement cards. Rolls of the three layers are prepared. A roll of lamination film serves as the base layer. Another roll of measurement membrane is then joined to one side of the lamination film. Lastly the top lamination film with windows is put on top of the measurement membrane. In this manner, a roll of viscosity measurement cards may be made. The roll may be cut to be packed in blister packs for daily use (for a Dysphagia patient staying at home) or as a roll (for use in hospitals and nursing homes where the daily requirements for use of the viscosity measurement cards is high).

While the foregoing detailed description discloses several embodiments of the present invention, it should be understood that this disclosure is illustrative only and is not limiting of the present invention. It should be appreciated that the specific configurations and operations disclosed can differ from those described above. It would also be appreciated that modifications will occur to others skilled in the art upon the reading and understanding of this specification and the annexed drawings. In particular regard to the measurement or indicator functions performed by the above described embodiments, the terms (including a reference to a “measurement indicator”) used to describe the viscosity measurement function of the invention are intended to correspond, unless otherwise indicated, to any component which performs the specified function of the described component, even though not structurally equivalent to the disclosed structure, which performs the function in the herein illustrated exemplary aspects of the invention.

### Advantageous Effects of the Invention

The Viscosity measurement indicator device of the invention is accurate and easy to use by checking the indicator markings which correspond to the viscosity levels under the IDDSI system. Technologies stated are therefore simple and requires little training of the food preparer. A very small volume of the liquid food (50 to 200 μL) would be needed for each sample as compared to 100 mL sample size which is the current standard for a sample of thickener food or liquid food) needed to carry out the viscosity measurement. The invention would reduce food waste and costs.

The costs of production of the viscosity measurement indicator device is low and hence its retail price would be low. In view of the need to take a viscosity measurement each time a liquid food is prepared for a Dysphagia patient, a number of viscosity measurements have to be taken each time a meal is prepared. The low costs of the viscosity measurement card and re-usability of the pipette would be advantageous to all users.

To wit,
the invention is simple to use
yet low in cost
gives fast and sure readings
a boon for those with swallowing difficulty
a great help to caregivers.

1. A viscosity measurement indicator device for measuring the viscosity of thickened liquids and liquid foods for Dysphagia patients using the International Dysphagia Diet Standardisation Initiative (IDDSI) Framework comprising three layers, namely a 1st layer, a measuring membrane layer and a 3rd layer, the 1st layer, the measuring membrane layer and the 3rd layer arranged or combined together to form the device, wherein a sample of thickened liquids or liquid food is taken and introduced to the viscosity measurement indicator device to show the viscosity level based on the IDDSI Framework.

2. The viscosity measurement indicator device as claimed in claim 1 wherein the indicator device is a card.

3. The viscosity measurement indicator device as claimed in claim 1 wherein the indicator device is a cylinder.

4. The viscosity measurement indicator device as claimed in claim 2 wherein the layer on top of the measuring membrane layer has an opening to allow introduction of the sample of thickened liquids or liquid food into the measuring membrane layer for measurement of viscosity thereof.

5. The viscosity measurement indicator device as claimed in claim 1 wherein the measuring membrane contains a hydrophilic substance which changes colour upon detection of water in the sample of thickened liquids or liquid food to be measured.

6. The viscosity measurement indicator device as claimed in claim 1, further comprising a 4th layer being a hydrophilic substance which changes colour upon detection of water in the sample of thickened liquids or liquid food to be measured.

7. The viscosity measurement indicator device as claimed in claim 1 wherein the length of the measuring membrane has Viscosity Indicators based on the IDDSI Framework calibrated to indicate a viscosity level from Level 0 to Level 4.

8. The viscosity measurement indicator device as claimed in claim 1 wherein the measuring membrane is made from paper.

9. The viscosity measurement indicator device as claimed in claim 1 wherein the measuring membrane is made from cloth.

10. The viscosity measurement indicator device as claimed in claim 1 wherein the measuring membrane is made from pure borosilicate glass.

11. The viscosity measurement indicator device as claimed in claim 1 wherein the measuring membrane is made from nitrocellulose membrane.

12. The viscosity measurement indicator device as claimed in claim 9 wherein the cloth is cellulose fiber.

13. The viscosity measurement indicator device as claimed in claim 9 wherein the cloth is alpha cotton cellulose.

14. The viscosity measurement indicator device as claimed in claim 9 wherein the cloth is linter fibers or cotton linter.

15. The viscosity measurement indicator device as claimed in claim 3 wherein the cylinder has a 1st layer being an outer surface and a 3rd layer being an inner surface of the cylinder, and the inner surface of the cylinder is treated with a layer of hydrophilic substance and the length of the cylinder has Viscosity Indicators based on the IDDSI Framework calibrated to indicate a viscosity level from Level 0 to Level 4.

16. The viscosity measurement indicator device as claimed in claim 15 wherein the inner surface of the cylinder is lined with a viscosity measurement layer treated with a hydrophilic substance and the length of the cylinder has Viscosity Indicators based on the IDDSI Framework calibrated to indicate a viscosity level from Level 0 to Level 4.

17. The viscosity measurement layer in the viscosity measurement device of claim 16 wherein the viscosity measurement layer contains a hydrophilic substance which changes colour upon detection of water in the sample of thickened liquids or liquid food to be measured.

18. The viscosity measurement indicator device as claimed in claim 16 wherein the viscosity measurement layer is made from paper.

19. The viscosity measurement indicator device as claimed in claim 16 wherein the viscosity measurement layer is made from cloth.

20. The viscosity measurement indicator device as claimed in claim 16 wherein the viscosity measurement layer is made from pure borosilicate glass.

21. The viscosity measurement indicator device as claimed in claim 16 wherein the viscosity measurement layer is made from nitrocellulose membrane.

22. The viscosity measurement indicator device as claimed in claim 19 wherein the cloth is cellulose fiber.

23. The viscosity measurement indicator device as claimed in claim 19 wherein the cloth is alpha cotton cellulose.

24. The viscosity measurement indicator device as claimed in claim 19 wherein the cloth is linter fibers or cotton linter.

25. A method to measure the viscosity of thickened liquids and liquid foods for Dysphagia patients using the International Dysphagia Diet Standardisation (IDDSI) Framework, comprising the steps of (i) introducing a sample of thickened liquids or liquid food to the viscosity measurement indicator device and (ii) observing dispersion action of the sample in the measurement membrane to indicate its viscosity level based on the IDDSI Framework.

26. The method to measure the viscosity of thickened liquids and liquid foods for Dysphagia patients using the IDDSI Framework as set forth in claim 25, using a viscosity measurement indicator cylinder wherein the cylinder with marker line thereon is dipped into a sample of thickener liquid or liquid foods, and said marker line is aligned with liquid level, and after several seconds, observing dispersion action of the thickened liquid upwards the cylinder to indicate its viscosity level based on the IDDSI Framework.

27. A kit to measure and adjust the viscosity of thickened liquids and liquid foods for Dysphagia patients using the International Dysphagia Diet Standardization (IDDSI) Framework comprising the viscosity measurement indicator device as claimed in claim 1, a pipette to introduce a sample of said thickened liquids and liquid foods into the viscosity measurement indicator card and a spoon for measuring water to be added to the thickened liquids and liquid foods for adjustment of the viscosity thereof.

28. A kit to measure and adjust the viscosity of thickened liquids and liquid foods for Dysphagia patients using the International Dysphagia Diet Standardization (IDDSI) Framework comprising the viscosity measurement indicator device as claimed in claim 1, a liquid dropper to introduce a sample of said thickened liquids and liquid foods into the viscosity measurement indicator device and a spoon for measuring water to be added to the thickened liquids and liquid foods for adjustment of the viscosity thereof.

29. The viscosity measurement indicator device for measuring the viscosity of thickened liquids and liquid foods for Dysphagia patients using International Dysphagia Diet Standardisation Initiative (IDDSI) Framework as set forth in claim 1, further comprising a device holder with an alignment hole, said device holder adapted to fit and hold onto the sides of the viscosity measurement card so that the alignment hole is directly over the opening of the measurement membrane for easy introduction of the thickened liquids and liquid foods onto the measurement membrane.
